(19) United States
(12) Patent Application Publication
(10) Pub. No.: US 2008/0082000 A1
(43) Pub. Date: Apr. 3, 2008

(54) Medical Camera

(76) Inventor: Michael Thoms, Bietigheim-Bissingen (DE)

Correspondence Address: FACTOR & LAKE, LTD, 1327 W. WASHINGTON BLVD., SUITE 5G/H, CHICAGO, IL, 60607, US

(21) Appl. No.: 11/596,701

(22) PCT Filed: Mar. 12, 2005
(86) PCT No.: PCT/EP05/05133

§ 371 Date: Jun. 26, 2007

(30) Foreign Application Priority Data
May 16, 2004 (DE) 10 2004 024 494.4

(51) Int. Cl. A61B 6/00

(52) U.S. Cl. 600/476

(57) ABSTRACT

A camera for use in the medical field includes a housing having an entrance window. Arranged around the entrance window are white light emitting LEDs and UV emitting LEDs. These LEDs are arranged under regular pitch. Behind the entrance window there is a colour filter absorbing light generated by the UV-LEDs. The light transmitted by this colour filter reaches an image converter. In an evaluating circuit an underground image is removed, and the result is displayed on a monitor.

The invention relates to a medical camera in accordance with the preamble of claim 1.

Such medical cameras having an electronic image converter are being more and more used in the medical field in view of rapidly representing medical findings and in view of thus obtaining more information or in view of being able to archive images at low costs and rapidly.

It is known that a plurality of bacteria shows fluorescence when being optically excited using light, which fluorescence can be a qualitative or even quantitative indication of an illness. The spectral properties of the fluorescence are determined by certain molecules which are found in bacteria and which are generated in the metabolism of bacteria. Thus porphyrins are generated as metabolic products in many bacteria which have absorption bands in the region of 350-450 nm (Soret-bands) as well as of 500-640 nm (Q-bands) as well as emission bands at 630 +−50 nm.

For detecting illnesses caused by such bacteria light is directed to the ill region having a wavelength suitable for the respective fluorescence molecule of the bacteria. The light emitted from the bacteria is detected using a detector being responsive only to light signals of the corresponding emission wavelength.

The object of the present invention is to provide a medical camera of the kind referred to above, which has a compact structure and which is suitable for the diagnosis of the surfaces of tissues forming part of a cavity of the human body, e. g. the mouth.

In accordance with the present invention this object is solved by a medical camera having the features given in claim 1.

With the camera in accordance with the present invention it is sure that the exciting UV light cannot directly reach the entrance window. Thus underground due to exciting light found in the image obtained will be small. Consequently also fluorescence light of small intensity will be represented so as to be well visible.

Advantages and further improvements are given in the subclaims.

With the further improvement of the invention in accordance with claim 2 one obtains the advantage that the UV light will be kept away from the image sensor, while all parts of the fluorescence light will be used for forming the image.

Edge filters as they are referred to in claims 3 and 4 are particularly useful for medical purposes.

Using a camera in accordance with claim 5 one obtains a high intensity of the UV light while only little heat is generated. The latter will cause an unpleasant feeling to the patient and could also result in damages of the tissue.

With the improvement of the invention in accordance with claim 6 homogeneous illumination of the field of view will be obtained.

The improvement of the invention in accordance of claim 7 allows to intensively illuminate an area to be diagnosed with UV light and to obtain a correspondingly high intensity of fluorescence light. At the same time, due to the duty-factor of the UV light source it is warranted that no damages of the tissue will occur.

In a camera in accordance with claim 8 the user can adjust the length and the succession of the individual light pulses generated by the UV light source in accordance with his needs.

A camera in accordance with claim 9 allows to study an area of tissue to be diagnosed also using white light, which is often desired in addition to the fluorescence study.

The further improvement of the invention in accordance of claim 10 is also made in view of homogeneous illumination of the field of view.

In a camera in accordance of claim 11 one obtains an image of the ill tissue regions of particularly rich contrast, since the part of the image forming an underground will be subtracted.

A camera in accordance with claim 12 allows to illuminate the area of tissue to be diagnosed using UV light of different wavelength. Thus there is an additional possibility of discerning between healthy and sick tissue or to discern regions of tissue showing different illnesses.

In this context the measure of claim 13 is again advantageous in view of obtaining an image showing good contrast.

The further improvement of the invention in accordance with claim 14 is advantageous in view of diagnosing regions of tissue which are difficult to access.

With the feature of claim 15 one has the advantage that the region to be diagnosed will be kept free from contaminants like blood.

In commercial cameras the optical system is formed as a telecentric optical system as is generally the case in cameras. In order to warrant good reproduction properties in such an optical system the lens arrangement facing the object, the intermediate lens arrangement and a lens arrangement being close to the image sensor must comprise a plurality (generally two) individual lenses. For this reason optical systems for such cameras are expensive.

A camera in accordance with claim 16 can be produced at lower costs while still showing good imaging characteristics.

In an optical system being non-telecentic in the case of medical cameras one can also use lens arrangements, which taken alone do not show particularly good imaging properties. More particularly, the individual lens arrangements may be formed by single lenses. Thus considerable cost savings are obtained and assembly of the optical system is simplified.

If the field diaphragm is selected as prescribed by claim 17, one has particularly good imaging characteristics. The third lens arrangement will be used in the central portion where the distortion errors are small. The intermediate lens arrangement will also be used in the marginal portions. However, it need not have surfaces of strong curvature so that the use of the marginal regions of this lens arrangement will not result in not acceptable distortion errors.

In accordance with claim 18 the first lens arrangement can be formed by a single optical component having very simple geometry.

The further improvements of the invention in accordance with claims 19 and 20 also cite simple ways to provide an intermediate lens arrangement and a lens arrangement being close to the image sensor.

A camera as defined in claim 21 is particularly well suited for use in the dental field, since the direction of observation is inclined with respect to the axis of the handpiece and may more specifically extend in a direction being perpendicular to the axis of the handpiece.

The further improvement of the invention in accordance with claim 22 is advantageous in view of ease of cleaning and sterilizing the camera.

A camera in accordance with claim 23 can be used as well for observing an object from the immediate neighbourhood as well as for observing an object from a larger distance. Using such a dental camera the doctor can, for example, register take pictures od details of a tooth or a general view of the teeth.

Claim 24 relates to a particularly simple way of adjusting the distance from which an image is taken.

In a camera in accordance with claim 25 the distance, from which an image is taken, can be adjusted without the need of a movable part extending through a wall of the housing of the camera.

The invention will now be described in more details refering to the drawings wherein:

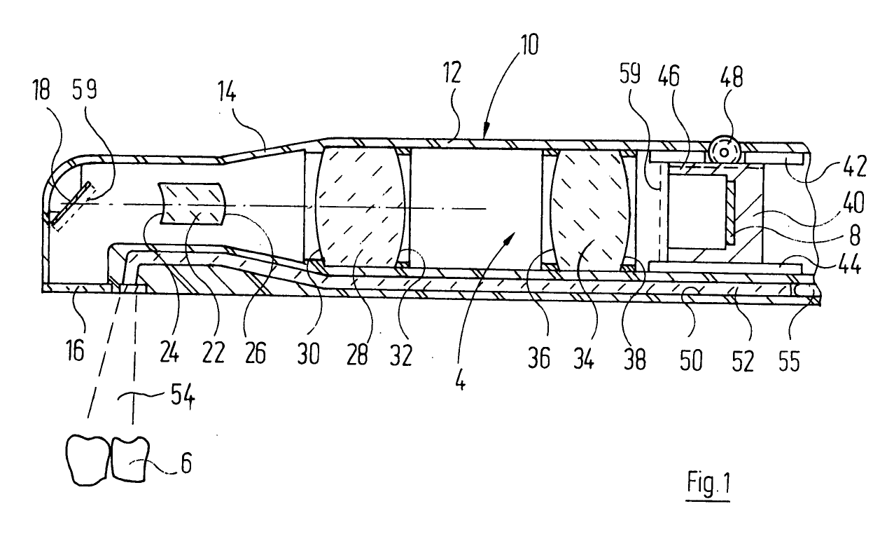

FIG. 1: Shows an axial section of a dental camera;

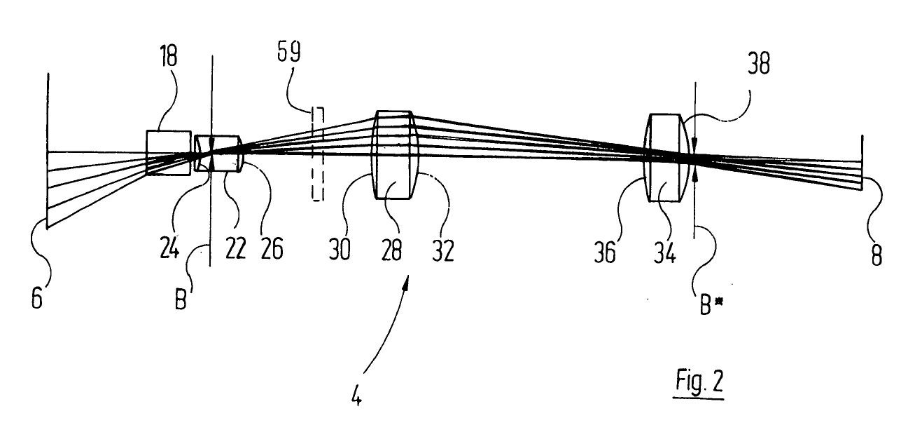

FIG. 2: Shows a schematic representation of the optical system of the dental camera in accordance with FIG. 1;

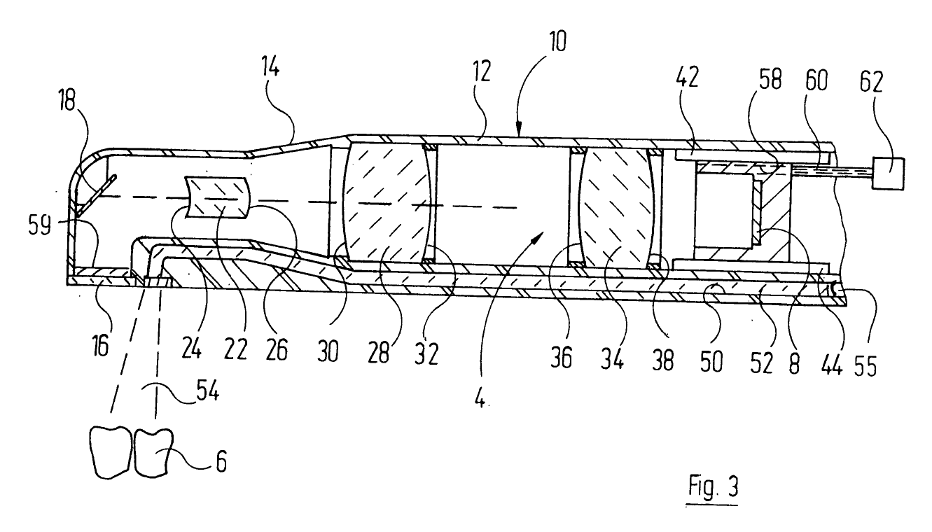

FIG. 3: Gives a view similar to the one of FIG. 1, wherein, a modified mechanical positioning system of the image converter is shown;

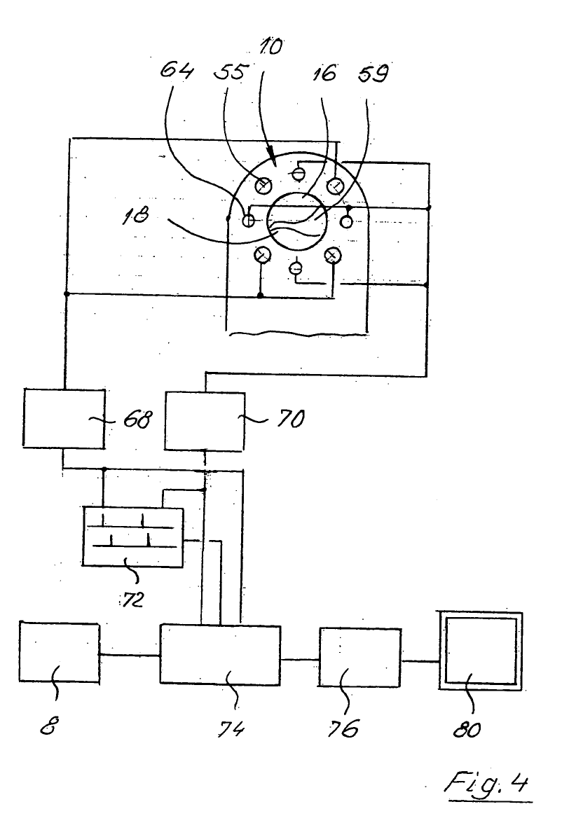

FIG. 4: Is a view of the upper side of a head of the diagnostic camera and a schematic representation of an operational unit co-operating with the image converter of the camera; and

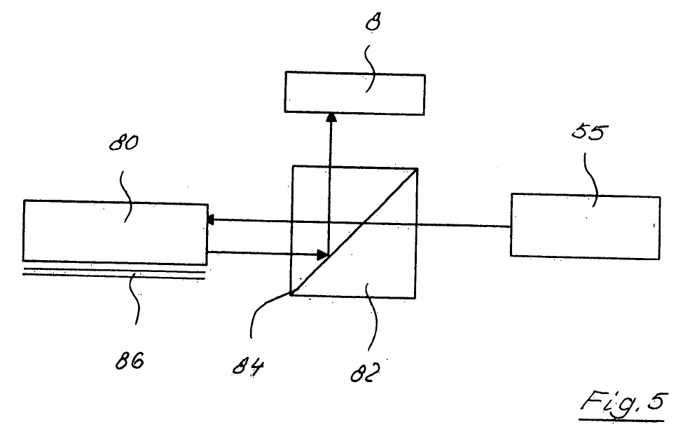

FIG. 5: Is a schematic representation of a modified camera which will be used at locations, where optical access is a problem.

The dental camera shown in the drawings has a housing 10 formed as a injection molded plastics part. The housing 10 is shown as being a one piece housing. It is to be understood that the specialist in the art can also use a multi-part housing as may be required by the respective production requirements. In such case the different parts of the housing can be connected in fluid-tight manner using seals or can be connected using an adhesive or can be welded.

The housing 10 has a grip portion 12 having essentially the form of a cylindrical sleeve being closed at one end thereof. At its free end the grip portion 12 carries a tapered and angled housing portion 14, the downward facing end of which is closed by an entrance window 16 and a light emission window 17 arranged in side-by-side relationship.

The entrance window 16 is simultaneously formed as an edge filter. It may be a coloured glass filter having an edge situated at about 550 nm, e.g. filter commercialized by ITOS Gesellschaft fur Technische Optik mbH as filter type OG 550. A coloured glass filter having an edge being closer to the wavelength of the UV light is for example the filter GG 495 of the firm Schott.

In the housing 10 there is arranged an optical system generally designated by 4. This optical system will form an image of a schematically shown object 6 (tooth or jaw) on an image converter 8. The image converter 8 can be provided in the form of a colour-CCD.

In the angled portion of the housing portion 14 there is arranged a deflection mirror 18 which is arranged under 45° with respect to the axis of the grip portion 12 and to the axis of the window 16. The deflection mirror can be also formed as a deflection prism, e. g. a right angle prism or a pentaprism.

Behind the deflection mirror 18 as seen in the direction of ray propagation, there is a lens 22 having a concave forward end face 24 and a convex rear end face 26.

Under a larger distance from the lens 22 there is arranged an intermediate lens 28, having a convex end face 30 facing the object and a convex end face 32 facing the image sensor.

A further lens 34 being adjacent to the image sensor is arranged under a major distance behind the intermediate lens. It has a convex end face 36 facing the object and a convex end face facing the image converter.

The image converter 8 is arranged on a carriage 40 running on guiding ribs 42, 44 formed on the interior surface of the housing 10 so as to be movable along the axis of the optical system 4. A tooth rack 46 is formed on one of the longitudinal surfaces of the carriage 40. It co-operates with a pinion 48 rotatably journalled in the housing 10 and having a pinion portion projecting through the housing 10 in outward direction. By rotating the pinion 48 the image converter 8 can thus be positioned along the axis of the optical system 4.

An essentially axially extending passage 50 is formed in the housing 10 to receive a light guide 52.

Behind the end of the light guide 52 being remote from the window 17 there is arranged a UW LED 55 emitting ultraviolet light having a wavelength between 390 and 410 nm. Such UV LEDs can be obtained e. g. by the firm ETG as type ETG-3UV400-30. The semi-conductor material used is InGaN which emits in the blue UW. A lens is integrated into the LED and as a result a very narrow light beam is obtained.

An end portion of the passage 50 and the light guide 52 are angled so that light applied to the light guide 52 will exit the light guide 52 in a direction being slightly inclined with respect to the axis of window 17, as shown at 54.

The image converter 8 and the light guide 52 are connected Os to image evaluating electronics by way of a connector arrangement not shown in the drawings (to be thought in the right portion of the figure).

The path of rays of the optical system 4 is shown in more detail in FIG. 2. For better clarity the situation is shown such as it would be in a straight line camera that is obtained from the camera in accordance with FIG. 1 if the deflecting mirror 18 formed as a deflecting prism was replaced by a flat parallel glass plate of same optical thickness and the window 16 was arranged on the axis of the grip portion 12. The various optical components have the same designations as in FIG. 1.

In addition various rays have been shown which extend from different points from the object 6 to associated points on the surface of the image converter 8.

One sees that with the optical system shown in FIG. 2 the filed diaphragm B* is conjugate to an image B lying in the neighbourhood of the lens 22. One recognizes that with such, position of the field diaphragm B and the image B thereof the lens 22 being arranged close to the object will be used essentially in the central region thereof, while the intermediate lens 28 is also used in marginal regions and the lens 34 being arranged adjacent to the image converter will be used only in the central region thereof.

Due to the shown arrangement of the three lenses wherein the intermediate lens 28 is spaced as well from the object facing lens 22 as from the lens 34 being adjacent to the image converter by a larger distance, the intermediate lens 28 need not have surfaces of strong curvature. Thus optical aberration is reduced. The fact that in the intermediate lens 28 marginal regions are used, too, will thus not result in a distortion of the image which is not acceptable.

The below table gives a concrete embodiment for putting into practice the optical system 4. The situation corresponds to the representation of FIG. 2.

In the table there are given, respectively, the number of the end face (reference numeral of FIGS. 1 and 2), the radius of curvature of the respective end face, the thickness of the layer of material following the end face and the kind of the optical medium (type of glass; A=air), which is arranged behind the respective end face. The last column cites the diameter of the respective end face. The length unit is always 1 mm.

| End face | radius | thickness | glass | diameter |
|---|---|---|---|---|
| object | ∞ | 9 | A | 13.21 |
| 19 | ∞ | 4 | SF8 | 3.80 |
| 21 | ∞ | 0.76 | A | 1.54 |
| 24 | −2.31 | 4.00 | N-LASF30 | 3.00 |
| 26 | −2.65 | 11.83 | A | 3.00 |
| 30 | 24.30 | 4 | N-LASF30 | 7.50 |
| 32 | −11.94 | 20.84 | A | 7.50 |
| 36 | 12.15 | 4.00 | N-ZK7 | 7.50 |
| 38 | −8.82 | 0.56 | A | 7.50 |
| diaphragm B* | ∞ | 15.13 | A | 0.98 |
| converter | ∞ | | | 4.85 |

Where in the column glass an “A” is given, the respective distances are distances, where the optical medium is air. The glass types correspond to the catalogue of optical glasses of the firm Schott.

The embodiment of FIG. 3 closely corresponds to the embodiment of FIG. 1. Corresponding components have been given the same reference numerals and will not be described in detail again.

In the embodiment of FIG. 3 the carriage 40 is provided with a threaded bore 58 receiving a threaded spindle 60. The threaded spindle 60 is driven by an electric motor 62 carried by the housing 10. Energizing lines of the electric motor 62 as well as connecting lines of the image converter 8 and the light guide 52 all extend to a connector to be thought in the right portion of FIG. 3 and providing a connection to a supply hose.

Thus the image converter 8 can be adjusted along the axis of the optical system 4 without the need of a mechanical acuating means extending through the wall of the housing 10.

In a modification of the embodiment described above the entrance window 16 can be formed as a fully transparent window and an additional colour filter 59 can be arranged on the deflecting mirror 18. This has the advantage, that the filter will be passed twice by the image light. As a still further modification the colour filter 59 can be arranged in front of the carriage 50 as shown in dashed lines or directly in front of the image converter 8.

In the embodiment of FIG. 4 components, which have already been described above refering to FIGS. 1 to 3 have again been given the same reference numerals. They need not be described in detail again.

Arranged around the circular entrance window 18 there are four white light LEDs 64. Situated between the latter there are four UV LEDs 66 also equally distributed in circumferential direction.

The white light generating LEDs 64 are connected to an output of an operating circuit 68 which will energize the white light generating LEDs selectively for continuous operation or for periods of operation.

In similar manner the UV generating LEDs 55 are connected with an operating circuit 70 energizing the UV LEDs for short spans of time, respectively.

Control of the operating circuit 70 is by means of a timer 72, which in addition to first and second activating pulses supplied to the operating circuit 70 and eventually 68 (if operated in intermittent mode as assumed here) will provide further control pulses being displaced in phase. These will thus be provided at times, wherein the UV LEDs (and eventually the white light generating LEDs if intermittently energized) will not be working.

The timing pulses and the control pulses of the timer 72 are supplied to a computing circuit 74. The latter has an input being connected to the output of the image converter 8. Each time that the timer 72 will receive a first activating pulse it will load from an image memory 76 co-operating therewith the fluorescence image integrated up to the respective moment and will add the image just received from the image converter 8 amplitudewise. Thereafter the thus obtained total image will be again stored in the image memory 76.

Each time when the timer 72 receives a second activating pulse it will load from the image memory 76 connected thereto a white light image integrated up to the respective moment and will add to this image amplitudewise the image just received from the image converter. Thereafter the total image thus obtained will be again stored in the image memory 76.

If the computing circuit 74 receives a control pulse it also loads the contents of the image memory 76 (fluorescence image and white light image) and will subtract therefrom the amplitudes of the image received from the image converter 8 and will store back the thus obtained new total image into the image memory 76.

One recognizes that thus the image memory 76 will contain a fluorescence image only showing the fluorescence due to the bacteria, the underground being formed by environmental light having been subtracted.

The same holds for the white light image.

The contents of the image memory 76 can be represented on a monitor 78.

In a variant of the embodiment of FIG. 4 the white light generating LEDs 64 can be replaced by further UV LEDs, which operate at a wavelength being different from the wavelength of the UV LEDs 66. Also one can provide as well white light diodes as a plurality of UV LEDs operating at different wavelengths. Control of the different LEDs is effected as described above.

Subtraction of the underground image is achieved for the further sets of UV LEDs as has been described above.

In addition one can additively or subtractively combine the images obtained with the different sets of the UV LEDs in view of making visible structures in the thick tissues.

In the embodiment of FIG. 5 components which have already been described above, are given the same reference numerals.

Only the most important components of the camera are shown. For diagnosing regions, where the optical access is very difficult, like a deep crevice, the paranasal sinus, the ear or the fissure of the tooth root a fibre optic system will be used instead of a lens optic system. Such is schematically shown at 80.

The UV LED 55 illuminates the rear end of the fibre optics 80 via a dichroitic beam divider 82 and a wavelength dispersive layer 84. The image light returning via the fibre optics 80 is supplied to the image converter 8 via the beam divider 82. The image converter 8 is only required to have one pixel for detecting the light and can be formed e. g. by a light sensitive diode or a photo transistor.

In the camera shown in FIG. 5 UV light will be directed to the region to be diagnosed via the layer 84 being transparent for UV light and the fibre optics 80.

The returning image forming light will run back through the fibre optics 80 and will reach the image converter 8 via the dichroitic layer 84.

If desired, the fibre optics 80 may comprise two separate fibre bundles one of which guides the UV light propagating towards the region to be diagnosed and the other of which guides the image forming light returning from the region to be diagnosed.

In the case of contaminations of the region to be diagnosed, e. g. contaminations in a fissure of the toothroot, it can be advantageous to keep contaminants like blood away from the region to be diagnosed. In such case a hollow fibre 86 can be provided extending parallel to the fibre optics 80 to pump a rinsing liquid to the region to be diagnosed.

In a further variant the fibre optics 80 can be designed such that it will simultaneously form a fluid passageway through which a rinsing liquid can be directed to the region to be diagnosed.

In such case the liquid is supplied to a transverse bore provided at a connecting end of the fibre optices.

Due to its high sensitivity the camera described above is well suited to make use of the autofluorescence of bacteria for discerning healthy and sick portions of tissue. It should be noted that this camera can also be used, when the bacteria have been marked by a fluorescence marker that has been additionally administered. Such markers are preferably supplied to the region to be diagnosed before the diagnosis is made, the marker being contained in a liquid solution. These markers will specifically accumulate at the bacteria to be diagnosed. If a fibre sensor camera is used, the fluorescence marker can be supplied before the diagnosis is made by a fluid passage way of this camera (cavities of these fibre optics or an additional hollow fibre).

The following further variants of the invention are feasable:

Since the intensity of the fluorescence may be very weak and since broad band interfering light may be present, e. g. due to other fluorescent cells or in the form of environmental light, it can be advantageous to use a plurality of photo detectors being sensitive at different wavelengths and to measure the intensity of the interfering signals as well as the intensity of the fluorescence signals to which the interfering signal is also super-positioned and to obtain the fluorescence signal in a subtracting step.

A further possibility to suppress an interfering signal due to environmental light can reside in providing the exciting light in the form of pulses and to integrate the returned intensity during this time and the decay time of the fluorescence signal. During a later interval preferably having the same length the intensity of the interfering light is integrated so that the fluorescence signal can be formed by combining the two precited signals in a subtractive way.

For example a region to be diagnosed is illuminated with an ultraviolett to blue semiconductor diode or semiconductor laser diode and the fluorescence is detected using a CCD-camera. To this end the illumination system of the camera described above will be provided with corresponding UV LEDs or laser diodes. A colour glass filter of the edge type (e. g. GG 495 Schott) is arranged in the optical path, which completely absorbs the exciting light and transmits the fluorescence light to the CCD-image converter. Environmental light or other interfering light can be eliminated in a way analogous to the method described above by intermittent provision of the exciting light and simultaneous and non-simultaneous detection of the fluorescence image and the image generated by the interfering light. Another possibility is to periodically switch to optical filters for detecting the intensity of the interfering light and the emission light, respectively.

Also, the image produced by the colour-CCD-image converter can be evaluated as to shifts in the colours.

In addition the fluorescence generated by the bacteria can be enhanced by certain substances, which are used to rinse the mouth before the diagnosis. So it is known for example from the photodynamic therapy of cancer that aminolevulinic acid and derivates of this acid are converted into porphyrins in cancer cells, which porphyrins are difficult to decompose in these cells. The same is true for many bacteria. Thus by rinsing the mouth with such a substance the fluorescence signal can be enhanced.

In the application described above using a camera the diagnosis of the complete space region can be made simultaneously. Thus incipient caries diseases at the surfaces of a tooth can be visualized.

However, it is also possible to visualize diseases of the skin like acne, which are caused by bacteria that can be activated optically, or melanoms of the skin, in which the concentration of porphyrin is clearly increased due to a higher rate of metabolism as compared to freckles.

In view of keeping interfering light away from the measuring region a cylindrical or conically formed cap may be arranged on the entrance window of the camera.

Since the currents of the CCD-image converter generated by the light intensities may be small as compared to the dark current of the image converter, a very sensitive detection by the CCD-image converter may require cooling thereof using e. g. a peltier element.

If a disease must be diagnosed in a crevice, where optical acces is a problem, e. g. the paranasal sinus, the ear, or a crevice of the tooth root, the region to be diagnosed is to be illuminated using an endoscope comprising a video camera as described above and an illuminating light source and the region to be diagnosed is filmed, or one limits the diagnose to a local study using a non-imaging system.

The latter may be conceived so as to direct the LED generated light or laser light to the region to be diagnosed using a light guide fibre. A second fibre may extend parallel to this fibre to receive the fluorescence emitted from the region to be diagnosed and transmitting the fluorescence light to a photodetector.

Also it can be advantageous in the case of measurements in a fissure in he a tooth root to keep the region to be studied free from contaminants like blood in view of having optical access to the region of diagnosis. In such case a further hollow fibre can be used to pump a rinsing liquid to the region to be diagnosed.

A further improvement of the invention results in using a single fibre for supplying the illuminating light and for receiving the fluorescence light. In such case a beam divider is provided at the coupling side of the laser light and the detecting side of the fluorescence light, respectively. The image divider may comprise a wavelength dispersive coating such that the blue laser light is transmitted, while the fluorescence light is reflected by 90°.

It is also possible to partly form the fibre as a hollow fibre such that the rinsing liquid is directed through this fibre to the surface to be studied. In such case the liquid must be supplied in the end portion of the fibre facing the laser by means of a lateral bore.

The diagnostic camera referred to above and constructed according to the teachings of the invention can also be used, if the bacteria are marked by an additionally applied fluorescence marker. Such markers are preferably supplied to the region to be studied as a liquid solution and will accumulate specifically at the bacteria to be studied. In the case of a fibre image converter being used the fluorescence marker can be supplied before the diagnosis via the rinsing fibre.

In the case of a high rate of accumulation of the fluorescence marker in the region of diagnosis it is also possible to detect not the fluorescence but the absorption of the fluorescence marker in the region of diagnosis.

Normally, this method of detection is less sensitive than the detection of the fluorescence. This alternative becomes possible by removing those optical components from the diagnostic apparatus which are responsible for colour separation of the emission signals.

Using this method it would be possible to also detect, e. g. discoloured dental tartar clinging to the root of a tooth in a tooth pocket. This method is appropriate e. g. for analyzing the tartar removal rate of a tooth. root in a vector treatment.

Since the absolute fluorescence signal depends amongst others from the coupling efficiency of the optical system, the distance, from which illumination is made, and the surface roughness as well as restoration materials in adjacent regions, the intensity of the disease cannot be easily determined from the absolute fluorescence intensity.

In such case a further improvement of the invention is appropriate. Therein the fact is used, that healthy tooth material also fluoresces under UV illumination. However, the emission spectrum is different. If a disease occurs, the emission spectrum is changed in that a part of the illumination light will be converted in the bacteria or the sick cells into the emission light of different wavelength. This change in the spectrum as compared to the healthy state can be detected using a colour sensitive image converter as a change in colour. This change in colour can be detected using a colour camera as a function of the spatial coordinates and can be represented on a monitor for showing the state of illness. Thus it is not the absolute intensity which is relevant for the diagnosis.

Accordingly, in accordance of the present invention the ratio between intensities of different spectral contributions can be generally used, to obtain a measure for the state of illness. Therein the one spectral contribution represents the reference signal that has not been modified by the disease and the other spectral contributions represents the signal in first line determined by the disease.

1. A medical camera comprising a housing, comprising a light source, comprising an optical system and comprising an image converter, wherein the housing has an entrance window for diagnosis light returning from the region to be diagnosed, said entrance window being not accessible directly by light generated by the light source, wherein the light source is a UV-light source and in that the entrance window is formed as a filter or that a filter is arranged in the optical path defined between the entrance window and the image converter.

2. The camera as in claim 1, wherein the filter is an edge filter.

3. The camera as in claim 2, wherein the edge of the filter is at a wavelength of 450 nm or at a longer wavelength.

4. The camera as in claim 3, wherein the filter edge of the edge filter is at 470 nm or a larger wavelength.

5. The camera as in claim 1, wherein the UV-light source comprises at least one UV-LED.

6. The camera as in claim 5, wherein the light source comprises a set of UV-LEDs surrounding the entrance window under equal pitch.

7. The camera as in claim 1, wherein the UV-light source is intermittently energized.

8. The camera as in claim 7, wherein the length and/or the distance of pulses controlling the intermittent energization is adjustable.

9. The camera as in claim 1, wherein a white light source is additionally provided.

10. The camera as in claim 9, wherein the white light source comprises a plurality of white light LEDs arranged around the entrance window under regular pitch and preferably energized intermittently.

11. The camera as in claim 7, wherein a timer in addition to first activating pulses for control of the UV-light source provides control pulses being phase shifted with respect to the first activating pulses and in that the first activating pulses and the control pulses are supplied to a computing circuit co-operating with an integrating image memory, the computing circuit upon receipt of the first timing activating signal adding the signal output from the image converter to the contents of the image memory while subtracting the image provided by the image converter from the integrated image contained in the image memory when receiving a control pulse.

12. The camera as in 1, wherein a plurality of UV-light sources operating at different wavelengths is provided.

13. The camera as in claim 12, wherein a timer provides first timing pulses for activating the first UV-light source and second timing pulses to activate the second UV-light source, the second timing pulses being phase shifted with respect to the first timing pulses, and will also provide control pulses, which are in phase to the two timing pulses, a computing circuit being provided receiving the two timing pulses and co-operating with an integrating image memory such that upon receipt of a first timing pulse viz. a second timing pulse the contents of the image converter is added to the contents of the image memory or subtracted therefrom and that upon receiving a control pulse the contents of the image converter is subtracted from the contents of the image memory.

14. The camera as in claim 1, wherein the optical system comprises a fibre optic system.

15. The camera as in claim 1, further comprising means for supplying a treatment fluid or a rinsing fluid to the region to be diagnosed.

16. The camera as in claim 1, wherein a diaphragm (B*) defining the field of view is provided and in that the image converter is arranged on the axis of the optical system, the optical system comprising a first lens arrangement being close to the object, an intermediate lens arrangement and a lens arrangement being close to the image converter and in that the diaphragm (B*) defining the field of view or an image (B) thereof is arranged in the region of the lens arrangement being close to the image converter.

17. The camera as in claim 16, wherein the diaphragm (B*) defining the field of view or an image (B) thereof is situated a small distance behind the lens arrangement being close to the image converter, which distance is about 2 to about 10% of the distance defined between the rear end face of the lens arrangement being close to the image converter and the light sensitive surface of the image converter.

18. The camera as in claim 16, wherein the lens arrangement being close to the object is formed by a lens being concavely/convexly curved.

19. The camera as in claim 16, wherein the intermediate lens arrangement is formed by a bi-convex lens.

20. The camera as in claim 16, wherein the lens arrangement being close to the image converter is formed by a bi-convex lens.

21. The camera as in claim 16, wherein a light deflecting means arranged in front of the lens arrangement being close to the object.

22. The camera as in claim 16, wherein an entrance window arranged in front of the lens arrangement being close to the object is connected to the housing in flush and fluid tight manner.

23. The camera as in claim 16, further including a means for positioning the image converter along the axis of the optical system.

24. The camera as in claim 23, wherein the positioning means comprise an actuating element extending through a wall of the housing.

25. The camera as in claim 23, wherein the positioning means comprise an electric motor which is energized by connector means which also provide an electric connection between the image converter and an electronic image evaluating unit.
